(19) United States
(12) Patent Application Publication
(10) Pub. No.: US 2004/0138560 A1
(43) Pub. Date: Jul. 15, 2004

(54) Real-time scan conversion and rendering of ultrasound data

(76) Inventor: Gianluca Paladini, Princeton, NJ (US)

Correspondence Address: Siemens Corporation, Intellectual Property Department, 170 Wood Avenue South, Iselin, NJ, 08830, US

(21) Appl. No.: 10/724,787

(22) Filed: Dec. 1, 2003

Related U.S. Application Data
(60) Provisional application No. 60/430,221, filed on Dec. 2, 2002.

(51) Int. Cl. A61B 8/00

(52) U.S. Cl. 600/437

(57) ABSTRACT

A 2D look-up table is created, where the 2D dimensions of the look-up table enclose the discrete space occupied by the angle spanned by the ultrasound vector data to be scan-converted. Each discrete i,j entry in the look-up table comprises 4 values: two fixed-point values (32-bit signed integer with 15 bits of fractional precision), one Boolean Flag, and one Integer Sum. The look-up table is used to convert (Y,Z) coordinates of the destination Cartesian volume to (R,Theta) coordinates of the source Cylindrical volume. (Y,Z) coordinates are used to access the i,j elements of the look-up table. The 2D look-up table is used for volume rendering, multi-planar reformatting or other imaging from data acquired in one format and output in another format. An additional look-up table may be used for further scan conversion along different dimensions, such as one LUT for scan conversion within each acquisition plane and another LUT for scan conversion across multiple acquisition planes.

### CROSS REFERENCE TO RELATED APPLICATION

This application claims the benefit of U.S. Provisional Application Serial No. 60/430,221, filed on Dec. 2, 2002, which is incorporated by reference herein in its entirety.

### BACKGROUND

The present invention relates to scan conversion, and more particularly to real-time scan conversion of ultrasound input data acquired in one format and output in another format.

Scan conversion is the process of converting data from Polar to Cartesian space, producing the rasterization of vector data onto a discrete Cartesian grid using interpolation and scaling. Volume Rendering is the process of projecting an arbitrarily oriented three-dimensional volume onto an image. Volume Rendering allows doctors to visualize internal anatomy in every direction.

As improvements are made in data acquisition, methods of scan conversion have proved to be inefficient, unable to deal with the vast amounts of data in a desirable time frame.

Scan conversion and Volume Rendering are particularly becoming important in the area of medical ultrasound three dimensional and four dimensional images. Such ultrasound applications have been frequently used by obstetricians when performing ultrasound examinations of pregnant mothers. The three dimensional ultrasound images provide the parents as well as the physician with a realistic view of the baby. Features such as the surface of the baby's face can be seen using these techniques and are very much in demand. However, in order to achieve acceptable images, the two dimensional data provided by the ultrasound transducer needs to be accurately and efficiently converted to three dimensional and four dimensional data. Many of the current techniques are inefficient and provide less than satisfactory four dimensional images.

Therefore, a need exists for a system and method for real-time scan conversion of 2D, 3D, and/or 4D data.

### SUMMARY

By way of introduction, the preferred embodiments described below include a method and system for scan conversion from an acquired ultrasound format to a display format. A 2D look-up table is created, where the 2D dimensions of the look-up table enclose the discrete space occupied by the angle spanned by the ultrasound vector data to be scan-converted. Each discrete i,j entry in the look-up table comprises 4 values: two fixed-point values (32-bit signed integer with 15 bits of fractional precision), one Boolean Flag, and one Integer Sum. The look-up table is used to convert (Y,Z) coordinates of the destination Cartesian volume to (R,Theta) coordinates of the source Cylindrical volume. (Y,Z) coordinates are used to interpolate the (R,Theta) values stored in (i,j) elements of the look-up table. This look-up table based scan conversion method is combined efficiently with 3D and 4D Volume Rendering and Multi-Planar Reformatting (MPR) methods to scan convert only visible data values, avoiding unnecessary scan-conversion of 3D and 4D data that does not contribute to the final image.

In a first aspect, a system is provided for scan converting ultrasound data from an acquisition format to a display format. A look-up table has values corresponding to a spatial conversion from the display format to the acquisition format. A processor is operable to identify acquired ultrasound data as a function of the values and operable to interpolate display values from the identified acquired ultrasound data.

In a second aspect, a method is provided for scan conversion of ultrasound data from an acquisition format to a display format. Acquisition format coordinates are identified with display format coordinates indexed to a look-up table. Acquired ultrasound data is determined from acquisition format coordinates interpolated from the look-up table. Display values are interpolated from the acquired ultrasound data determined using the acquisition format coordinates identified from the look-up table.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a diagram of a system according to an embodiment of the present invention;

FIG. 2a is a diagram of 4D ultrasound data according to an embodiment of the present invention;

FIG. 2b is a diagram of an (R,Theta) plot corresponding to ultrasound data acquired using a mechanical wobbling transducer according to an embodiment of the present invention;

FIG. 3 is a flow chart of a method according to an embodiment of the present invention;

FIG. 4 is a diagram of an (R,Theta) plot superimposed on a Cartesian coordinate system according to an embodiment of the present invention;

FIGS. 5a and 5b are illustrations of a ray caster for Volume Rendering and MPR according to embodiments of the present invention; and

FIGS. 6a and 6b are illustrations of a spherical section of an (R, Theta, Phi) coordinate system according to an embodiment of the present invention.

### DETAILED DESCRIPTION OF PREFERRED EMBODIMENTS

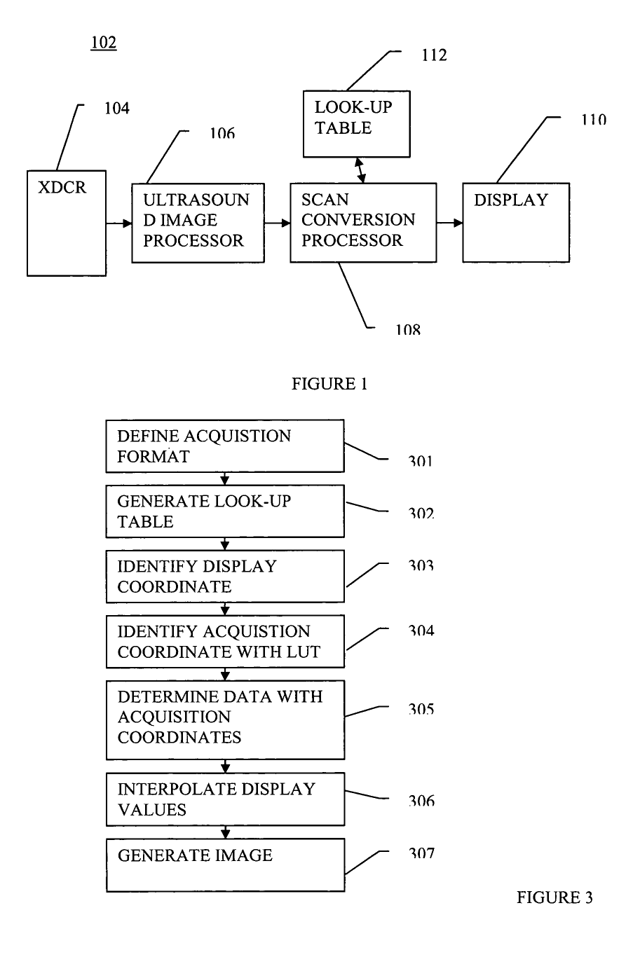

FIG. 1 shows an ultrasound system 102 for scanning a patient in an acquisition format and displaying an image in an image format. The ultrasound system 102 includes a medical sensor, such as a transducer 104 with an ultrasound image processor 106. The ultrasound transducer 104 is used to scan a patient and collect data which is then converted to an ultrasound image which is shown on display 110.

In a typical ultrasound system, the scanned data from the transducer 104 is provided as a two dimensional image. Any now know or later developed transducers 104 may be used, such as PZT or CMUT elements in a one or multidimensional array. In one embodiment, the transducer 104 is a wobbler transducer. For example, the transducer 104 includes a motor that causes the transducer 104 to wobble over the patient in a back and forth motion. The result is several two dimensional φ planes that are converted from Polar coordinates to three dimensional Cartesian volumes which are then volume rendered to produce 3D and 4D images as will be described in greater detail hereinafter.

The ultrasound image processor 106 includes beamformers, detectors, filters and/or other components for acquiring ultrasound data with the transducer 104. The ultrasound image processor 106 scans the patient with acoustic energy in one or more formats, such as sector, Vector® or linear. Other two or three dimensional scanning formats may be used. For example, the acquired ultrasound data represents a volume in the acquisition format. The acquired ultrasound data is formatted along a Polar coordinate system.

In one embodiment, the ultrasound image processor 106 includes a two-dimensional scan converter for converting ultrasound data to a Cartesian coordinate system for two-dimensional imaging. The ultrasound sound image processor 106 outputs acquired ultrasound data associated entirely with the Polar coordinate format or outputs frames of data associated with two-dimensional scan conversion to a Cartesian format with each frame of data positioned relative to each other in the Polar coordinate format.

The scan conversion processor 108 is any now known or later developed processor, such as a general processor, application specific integrated circuit, digital signal processor, digital circuit, analog circuit or combinations thereof. In one embodiment, the scan conversion processor 108 is a graphics processing unit, such as provided on a video card. For example, the graphics processing unit includes a vertex processor, an interpolator, a fragment processor and a display buffer. The scan conversion processor 108 is operable to identify acquired ultrasound data as a function of acquisition format values and operable to interpolate display values from the identified acquired ultrasound data. For example, the scan conversion processor 108 is operable to determine display coordinates of interest for a plane in a volume or for a plurality of rays through the volume. The acquired ultrasound data is identified by inputting the display coordinates of interest into a look-up table.

According to preferred embodiments, a look-up table based method is used for real-time Scan Conversion ultrasound input data from Polar coordinates to Cartesian coordinates, to generate raster data and render such data as 2D images, 3D volumes, and/or 4D volumes (3D volumes+Time) onto a computer display using Volume Rendering and Multi-Planar Reformatting (MPR). The look-up table 112 is a memory or buffer. The look-up table has values corresponding to a spatial conversion from the display format to the acquisition format, such as storing Polar coordinates indexed by Cartesian coordinates. In a further embodiment, the display coordinates also index other values in addition to fixed point coordinates (e.g., R and Theta values of the acquisition format), such as a Boolean Flag indicating whether the data set corresponds to a location outside the scanned region and/or an integer sum for other processing.

In one embodiment, the look-up table 112 is a video memory of a graphics processing unit. The acquired ultrasound data is loaded as a 3D texture and the spatial conversion look-up table is stored as an OpenGL or other texture. A virtual Cartesian volume is rendered and multiple planes orthogonal to a viewing direction are identified as polygons. The polygons are rasterized into fragments. Each fragment is associated with a three dimensional texture coordinate. The three dimensional texture coordinate is input into the look-up table to interpolate a Polar coordinate. The present invention allows real-time Scan Conversion of ultrasound data, thereby enabling rapid Volume Rendering of 4D data acquired in real-time by an ultrasound imaging apparatus, i.e., time sequences of 3D volume data.

The look-up table 112 accounts for other spatial variation in additional embodiments. For example where the acquired ultrasound data is acquired with a wobbler transducer array, the Polar coordinate values of the look-up table include corrections for shear distortion. In one embodiment, an axis of the acquisition format and the display format are defined in common, so only a two-dimensional look-up table is used. As another example, where the acquired ultrasound data represents a plurality of scan planes with the acquired ultrasound data of each scan plane in a Cartesian coordinate format but each of the scan planes positioned in the volume in a Polar coordinate format, the look-up table values correspond to the spatial conversion from the Cartesian coordinate format to the Polar coordinate format relative to the scan plane positions in the volume. As yet another example, the look-up table 112 is a two-dimensional look-up table of values corresponding to the spatial conversion from the display format to the acquisition format for at least one acquisition plane. An additional two-dimensional look-up table corresponding to spatial conversion from the display format to the acquisition format along different dimensions is also provided. The second look-up table replaces the functionality of the hardware scan-converter in one embodiment. Instead of using the scan-converted frames 202 aligned within the acquisition format, the acquired (R′,Phi) data for each frame 202 is scan converted with the additional LUT. The height of the resulting scan-converted frame becomes the radius R of the (R, Theta) wobbling direction. The additional two-dimensional look-up table may be combined with the two-dimensional look-up table to provide a three-dimensional look-up table.

The display 110 is a CRT, plasma screen, LCD, projector or any other now known or later developed display device. The display 110 is operable to generate one or more images, such as a two-dimensional representation of a three-dimensional volume (e.g., a Volume Rendering) that is static or varies as a function of time (e.g., four dimensional imaging). A two-dimensional image of a scanned plane or an arbitrary plane within a volume, also known in the art as a Multi-Planar Reformatting (MPR) image, may additionally or alternatively be displayed. The display 110 has a display format, such as a Cartesian format of vertical and horizontal lines. Each pixel on the display is generated as a function of display values, such as a grey scale intensity level, RGB color value, YUV color value or other display information. In one embodiment, an RGBA look-up table addressed by other display values (e.g., intensities) is provided to generate each pixel. The RGBA look-up table operable to output an RGBA value corresponding to the display value.

The present invention can be used as, for example, a preprocessing step to scan-convert all the data, or it can be used ‘on-the-fly’ in Volume Rendering and MPR techniques, so that only the visible portions of the data are scan converted. For example, the display values for the image are a function of alpha blending of a plurality of acquired ultrasound data values. The scan conversion processor 108 is operable to limit a number of acquired ultrasound data values blended as a function of a threshold such that scan conversion of occluded ultrasound data values is avoided. After each additional alpha blending, the alpha value buffered is compared to the threshold. If the alpha value exceeds the threshold, sufficient opacity is represented by the alpha value, so further scan conversion of data samples along the associated ray line is avoided.

It is to be understood that the present invention may be implemented in various forms of hardware, software, firmware, special purpose processors, or a combination thereof. In one embodiment, the present invention may be implemented in software as an application program tangibly embodied on a program storage device. The application program may be uploaded to, and executed by, a machine comprising any suitable architecture. For example, the scan conversion processor 108 and look-up table 112 are embodied in a workstation remote from the ultrasound image processor 106.

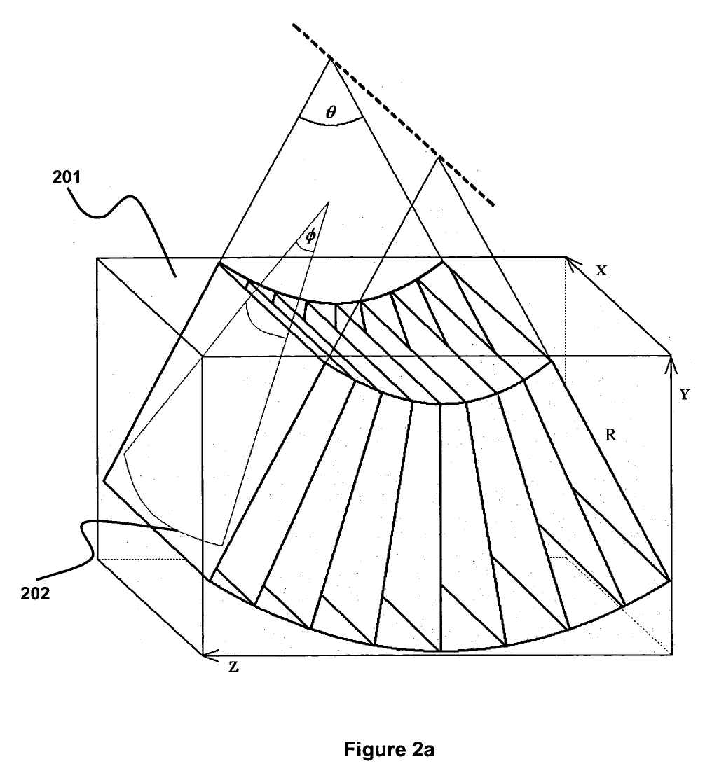

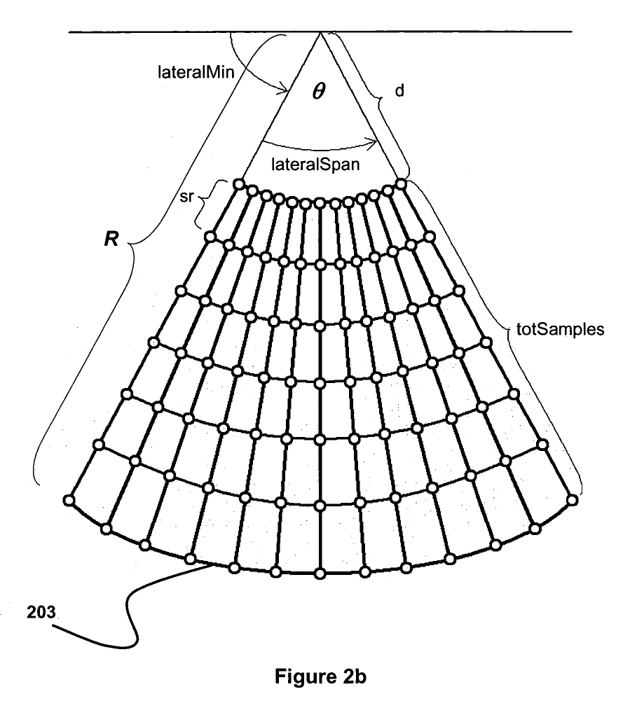

Referring to FIGS. 2a and 2b, the volume 201 represented by acquired data is created by the ultrasound transducer wobbling or scanning over the object of interest. The acquired data may represent an array of scan-converted frames 202 arranged in a Cylindrical coordinate system, e.g., the hardware scan converter has been used to generate 2D (I,J) frames of the (R,Phi) space but it has not scan-converted them along (R,Theta) to generate an (X,Y,Z) Cartesian volume. It is assumed that the Cylindrical coordinate system is X,R,Theta, where X is aligned with the I axis of the scan-converted frames, that the voxel data is valid within a Cylindrical section bounded by R and Theta ranges 203, and that the Cartesian volume represents the X,Y,Z boundaries of the (X,R,Theta) Cylindrical volume. Alternatively, the acquired data represents data entirely in a Polar coordinate (e.g., Cylindrical volume) format.

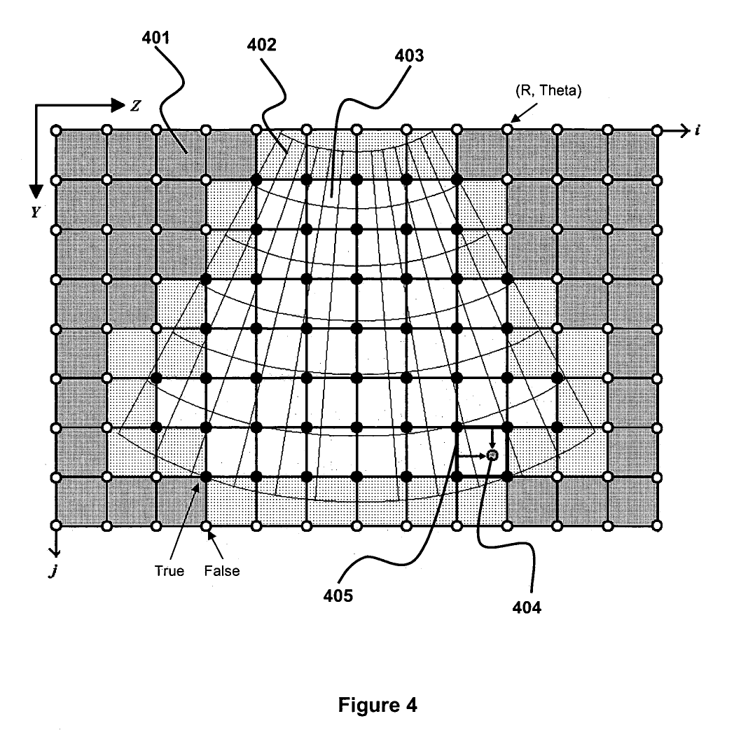

FIG. 3 shows a flow chart of one embodiment that generally illustrates a method for scan converting from an acquired ultrasound format to a display format. The method may enable rapid Volume Rendering of the data to provide 3D or 4D images or rapid selection and rendering of an MPR image representing a plane within the volume. The cylindrical coordinate system described above is used to provide fast scan conversion of the ultrasound Cartesian coordinate data. The user or system defines the acquisition format in act 301. For example, the user configures an imaging system to scan over a particular range of angles and obtain samples representing a depth from a beginning depth to an ending depth. The range of angles is provided by electronic steering, such as within a single scan plane or between scan planes, or is provided by mechanical steering, such as the sweep range of a wobbler transducer array.

In act 302, the look-up table is created. The look-up table is generated as a function of a spatial relationship of a display format with the user configured acquisition parameters (i.e., the acquisition format). For example, prior to acquisition but after configuring the system for acquisition, the spatial look-up table is created. In one embodiment, a 2D look-up table is created, where the 2D dimensions of the look-up table enclose the discrete space occupied by the angle spanned by the ultrasound vector data to be scan-converted. The two-dimensional look-up table is populated with acquisition format coordinates for each coordinate of a Cartesian volume. Each discrete (i,j) entry in the look-up table comprises 4 values: two fixed-point values (e.g., 32-bit signed integer with 15 bits of fractional precision), one Boolean Flag, and one Integer Sum. In other embodiments, different values are used, such as just the two fixed-point values.

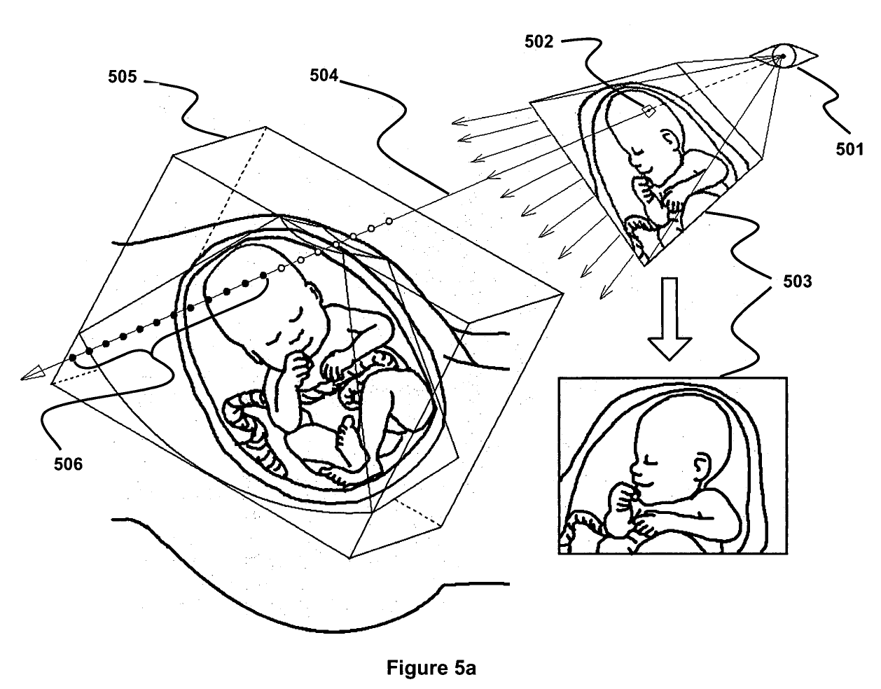

As shown in FIG. 4, the two fixed-point values represent the coordinates in normalized (R,Theta) space corresponding to the display format coordinates. The Boolean Flag entry is a byte value in the range 0-1 (0=False, 1=True) indicating whether each i,j entry corresponds to a location outside of the spanned angle Theta. The Integer Sum entry comprises the sum of the 2×2 neighborhood of Boolean Flags at each i,j look-up table location, in order to determine if the 2×2 neighborhood is an empty cell 401, a partially empty cell 402, or a completely full cell 403. An arbitrary (R, Theta) coordinate 404 is bilinearly interpolated using the nearest four (R, Theta) values 405 stored in the look-up table.

In the special case in which the Polar coordinates are symmetrical with respect to the middle vertical axis of the image (ranging from −Theta/2 to +Theta/2), only half of the look-up table entries may be computed using atan2, sqrt and division operations in the programming “C” language. Other programming languages may be used. The other half of the look-up table is a mirror of the values computed for Theta/2 and may be computed with simpler add/subtract operations.

The look-up table entries are initialized. Assuming that the ultrasound data has been acquired starting at a lateral angle lateralMin, and spanning over an angular range lateralSpan, the start and stop angle values are therefore:

startAngle=lateralMin;

stopAngle=lateralMin+lateralSpan.

The use of a start and stop angle are a more flexible representation of the spanned angle Theta. The half field of view, fov2, enclosing the start and stop angles is determined as:

absStart=abs(startAngle);

absStop=abs(stopAngle); and

fov2=(absStart>absStop) ? absStart:absStop

Without loss of generality, world units are assumed to be in millimeters and angles are in radians. Given the value d as the distance in world units from the apex of rotation (also known as Radius of Curvature) to the first location for which data is acquired, given totSamples as the total number of samples along an ultrasound data vector, R, and given sr as the scaling factor converting totSamples into a vector length in world units, then the dimensions xDim, yDim in world units of the Cartesian space enclosing the spanned angular range can be determined as:

xDim=radius*sin(fov2)*2.0;

yDim=radius−(d*cos(fov2)); and

where radius=d+(totSamples*sr).

Radius and d are converted to raster coordinates rs and ds, respectively, as follows:

if d is nonzero:

rs=(radius*totSamples)/(radius−d); and

ds=rs−totSamples.

otherwise, if d is zero:

rs=totSamples; and

ds=0

The raster width and height dimensions the spanned angular range can be determined as:

hRes=rs*sin(fov2)

rsMin=ds*cos(fov2)

if rsMin is less than zero, then rsMin=0

width=integer_of((hRes*2.0)+0.5)

height=integer_of((rs−rsMin)+0.5)

The 2D look-up table can be allocated using a resolution xRes,yRes equivalent to the width and height determined above:

xRes=width

yRes=height

An arbitrary look-up table resolution xRes,yRes can also be specified directly as a parameter, instead of being computed. In that case, scaling factors sx and sy need to be adjusted to compensate for the change in resolution:

sx=width/xRes

sy=height/yRes

The look-up table is allocated with dimensions xRes,yRes. Then, each element of the look-up table is initialized as follows:

startAngle=startAngle+fov2

stopAngle=stopAngle+fov2

fov=fov2*2

st=1.0/(stopAngle−startAngle)

sr=1.0/(rs−ds)

for each element i,j of the look-up table

x=(j*sy)+rsMin

y=((FLT64)i*sx)−hRes

r=sqrt(x*x+y*y)

theta=atan2(y, x)+fov2

lut[j][i].validRange=0 if (r<ds) or

(r>rs) or (theta<startAngle) or

(theta>stopAngle), otherwise it is equal to 1

lut[j][i].r=fix((r−ds)*sr)

lut[j][i].theta=fix((theta−startAngle)*st)

lut[j][i].flagSum=0;

where lut[j][i].validRange is the Boolean flag indicating if the Cartesian i,j look-up table location corresponds to an r,theta value within the spanned field of view angle, lut[j][i].r is the radius at location i,j normalized in the range 0 to 1, lut[j][i].theta is the theta angle at location i,j normalized in the range 0 to 1, and fix( ) is a function converting floating point values to a fixed-point integer representation, so that the look-up table values can be used with fast integer math. In this first pass, lut[j][i].flagSum is initialized to zero. A second pass is used to initialize all the i,j flagSum elements of the look-up table, computed as follows:

for each element i,j of the look-up table, except for elements i=xRes−1 and j=yRes−1:

lut [j][i].flagSum=lut[j][i].validRange+

lut[j][i+1].validRange+

lut[j+1][i].validRange+

lut[j+1][i+1].validRange.

The look-up table described in the present invention is used to convert (Y,Z) coordinates of the destination Cartesian volume to (R,Theta) coordinates of the source Cylindrical volume. In this case (Y,Z) coordinates are used to access the i,j elements of the look-up table. After or in parallel with the generation of the look-up table, ultrasound data is acquired using the acquisition format. For example, the ultrasound data is acquired with a wobbler transducer array.

Before, during or after acquisition of the acquired ultrasound data, the display coordinates of interest are determined in act 303. For example, the display coordinates of interest correspond to a plane through the volume, such as an arbitrary plane of MPR. As another example, the display coordinates of interest for a plurality of rays or cut planes through the volume are identified for Volume Rendering.

In act 304, the acquisition coordinates corresponding to the identified display coordinates are identified with the look-up table. For example, the Cartesian coordinates or display coordinates of interest are input into the look-up table. Polar coordinates are output from the look-up table in response to the input. In one embodiment, the acquired ultrasound data represents a plurality of scan planes with the acquired ultrasound data of each scan plane in a Cartesian coordinate format and each of the scan planes positioned in the volume in a Polar coordinate format. The look-up table is used to spatially converting coordinates from the Cartesian coordinate format to the Polar coordinate format relative to the scan plane positions in the volume.

The boundaries of the Cartesian volume are defined as “virtual” because there is no actual Cartesian data inside the volume, and its Y,Z dimensions are considered to be the same as the xRes,yRes dimensions of the look-up table. Using a destination-driven approach, for every X,Y,Z coordinate of the volume, it is possible to convert Y,Z to R,Theta using the look-up table entries. The X coordinate can be kept unmodified if the X resolution of the volume is the same as the I resolution of the frames in Cylindrical space. If the resolution is different, a scaling factor can be used to convert X to X′.

In one embodiment, the look-up table values (e.g., Polar coordinate values) are corrected to account for shear associated with obtaining ultrasound data with a wobbler transducer. As an alternative, the coordinates output by the look-up table are corrected or altered. The value of X may be altered to compensate for the wobbling motion of the transducer while the frames in Cylindrical space are acquired. Since each frame comprises data vectors that are sampled while the transducer is wobbling, a shear distortion occurs. Such shear distortion due to the wobbling motion can be corrected by modifying the Theta value as a function of X, such that:

Theta′=kt*(Theta−(kx*X))

where kt and kx are determined as:

dtMax=(1/totVectors)*kwc

xConv=1/(frameWidth−1)

tWidth=1−dtMax

kx=dtMax*xConv

kt=1/tWidth

where kwc is a normalized wobbling correction factor from 0.0 to 1.0 which controls the amount of shear correction that should be applied in order to compensate for the wobbling motion (FIG. 3, Block 306). The coefficients kt and kx can be stored in fixed-point notation, so that Theta′ can be determined with integer math using:

Theta′=fix(kt*(Theta−fix(kx*X))).

In act 305, the acquired ultrasound data is determined as a function of the acquisition coordinates output by the look-up table. The acquired ultrasound data to be scan converted or used for rendering is indexed in the acquisition format. Since the display coordinates of interest are converted by the look-up table to acquisition format coordinates, the desired ultrasound data values are obtained.

One way to scan-convert the Cartesian volume is to perform the conversion process for all data: loop over all the X,Y,Z coordinates of such volume, use the Y,Z coordinates as i,j indices into the look-up table elements to find the corresponding R,Theta coordinates, correct Theta to Theta′ to compensate for shear distortion due to wobbling motion, then use X,R,Theta′ coordinates to access the corresponding ultrasound data values.

In act 306, the acquired ultrasound data is interpolated to display values. Since X,R,Theta values are floating point values (or fixed-point values, depending on the chosen representation) not located at discrete location, an interpolation step, such as trilinear interpolation, is used to determine an accurate data value at the computed X,R,Theta location. The resulting interpolated Cartesian volume data can then be rendered using Volume Rendering and Multi-Planar Reformatting algorithms known in the art.

In act 307, an image is generated from the interpolated ultrasound data. The display value represented by each pixel of the display grid corresponds to the interpolated ultrasound data identified using the look-up table. In one example, a two-dimensional image representing a scan plane is generated. In another example, a two-dimensional image representing an arbitrary plane is generated in the display format as a function of the interpolated display values. As yet another example, a two dimensional representation is generated as a Volume Rendering. The Volume Rendering is performed as a function of the display values. The Volume Rendering is static or dynamic, such as volume rending as a function of time.

The approach of scan-converting all the data is not efficient for Volume Rendering and for Multi-Planar Reformatting. In Volume Rendering, depending on the projection method used, there might be some data values which are not visible, because they are occluded by other opaque parts of the volume as projected from a particular viewing direction. In such case, unnecessary look-up table conversion and interpolation of data values which are not visible may be avoided. In the case of Multi-Planar Reformatting, which involves the computation of a planar slice of the Cartesian volume, only the data values on the MPR slice are needed, and therefore only the data points which intersect the MPR plane are scan converted.

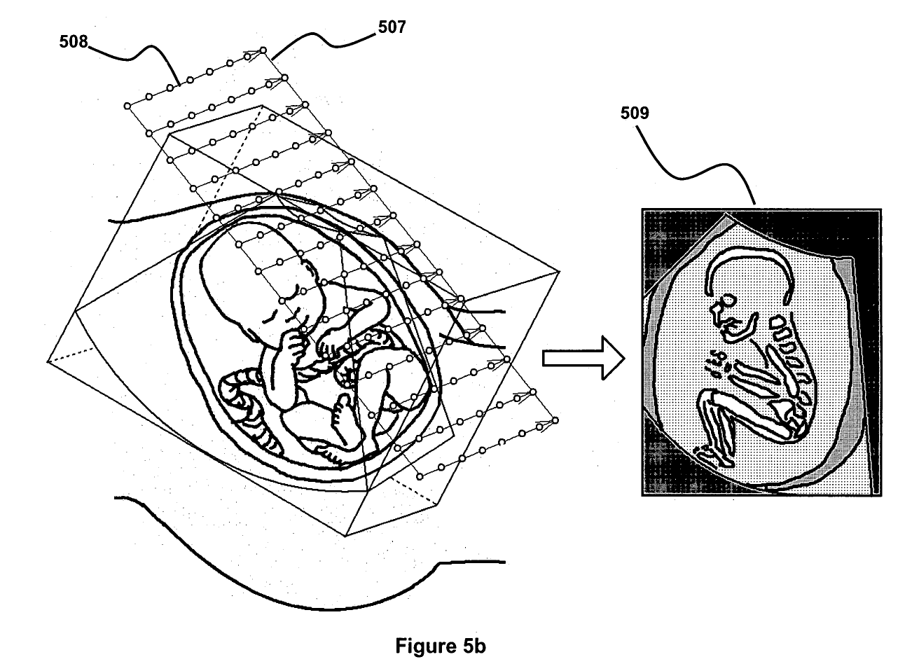

One preferred embodiment of this invention associates the use of the scan conversion look-up table with a Volume Rendering technique known in the art as Ray Casting and represented in FIG. 5a. In Ray Casting, rays are cast from an arbitrary observer location 501 through the pixels 502 of a two-dimensional Volume Rendering image 503, and a plurality of acquired ultrasound data values along each ray is alpha blended for each of the pixel values.

Referring to FIG. 5a, in a Ray Caster, each ray 504 is first clipped against the boundaries of the “virtual” Cartesian volume 505. Whether the acquisition format coordinates are outside of scanned region is identified with the look-up table. X,Y,Z fixed-point coordinates are determined along the clipped rays, and the look-up table is used to convert (Y,Z) to (R,Theta). The integer part of the (Y,Z) coordinates is determined, which correspond to an entry i,j in the look-up table. If the pre-computed sum of the Boolean flags at location i,j is 0 (i.e. lut[j][i].flagSum=0), then the Cartesian voxel value is located in empty space, and the sample can be skipped (space-leaping optimization). Otherwise the fractional part of the (Y,Z) coordinates can be used it to bilinearly interpolate the four neighbors and computing the corresponding (R,Theta) value at that location. It takes 6 fixed-point linear interpolations to compute such value. If lut[j][i].flagSum<4, the method determines whether the interpolated (R,Theta) coordinate is out of range, since it is near the boundaries of the valid (R, Theta) range. Otherwise if the sum is 4, the (R,Theta) values are assumed to be within range. If wobbling correction is needed, then Theta′ is determined, otherwise Theta′=Theta.

Since X and I are parallel, the (X,R,Theta′) fixed-point coordinates can be used to trilinearly interpolate the Cartesian voxel value from the Cylindrical data. The integer part of (X,R,Theta′) can be taken and the 8 nearest neighbors determined. The fractional part can be used to perform the trilinear interpolation.

Once the voxel value has been interpolated, the rest of the ray casting program works the same as if the volume data came from a Cartesian volume. For example, the display values (e.g., results of the interpolation) are input into an RBG look-up table. RGB values corresponding to the display values are output in response to the input.

The number of acquired ultrasound data values blended or ray cast may be limited as a function of a threshold. Scan conversion of all of the acquired data is avoided due to application of the threshold. For example, the voxel values (e.g., display values) are used to look-up a corresponding RGBA value in a Transfer Function look-up table. The RGBA value is alpha-blended with an OVER operator, and the accumulated alpha is checked. If the accumulated alpha value has reached full or another threshold opacity, scan-converting along the ray is discontinued (early-ray termination optimization) since the portion omitted 506 is not rendered.

Alternatively, this approach of sampling along an arbitrarily oriented ray can be also used to generate an arbitrarily oriented cross-section 507 known in the art as an MPR (Multi-Planar Reformatting) image, as illustrated in FIG. 5b. In such case, the method does not alpha blend samples along the ray, but rather considers each ray 508 to be a scanline of an MPR image 509, and stores the samples along the ray as pixel values.

In an another embodiment, optimization may be more optimal but memory intensive. Each ray sample which gets trilinearly interpolated is saved in a 3D look-up table. The X and (R,Theta) values which were bilinearly interpolated from the 2D look-up table are saved. The 3D look-up table size is the number of rays casted for the output (N×M) image buffer, times the number of samples per ray. If the viewing direction does not change from frame to frame, subsequent frames can take advantage of the 3D look-up table and render the final image faster.

Even if a scan-conversion of the entire Cartesian dataset is not particularly efficient, it might have some advantages. One advantage is that the scan-conversion process can have a better data coherency than in ray casting. One difference is that the rays can now be considered all parallel to the X axis, which corresponds to the I axis of the scan-converted frames. When samples are determined along the same scanline direction as the frame data is stored, the CPU cache is utilized in an optimal way, and many values used to scan-convert coordinates will be reused from the cache for subsequent samples. Where the resampling does not go across slices, cache coherency is maintained. Loss of cache coherency may result in scan conversion that is as much as 4 times slower. Also, since all the X coordinates correspond to I coordinates, there is no need to use the fractional part for X, and the voxel interpolation is reduced to a bilinear operation (unless a different X resolution is chosen for the final output).

By adjusting the size of the (R,Theta) look-up table and perhaps using an additional look-up table for the X axis, it will be possible to use the same approach to scan convert volumes at arbitrary resolutions. This approach can be used to render volumes at different frame rates, by varying the resolution of the scan-conversion.

As an alternative to Ray Casting, Shear-Warp rendering can be used to perform a similar look-up table-based scan-conversion on the fly, during the Shear projection step.

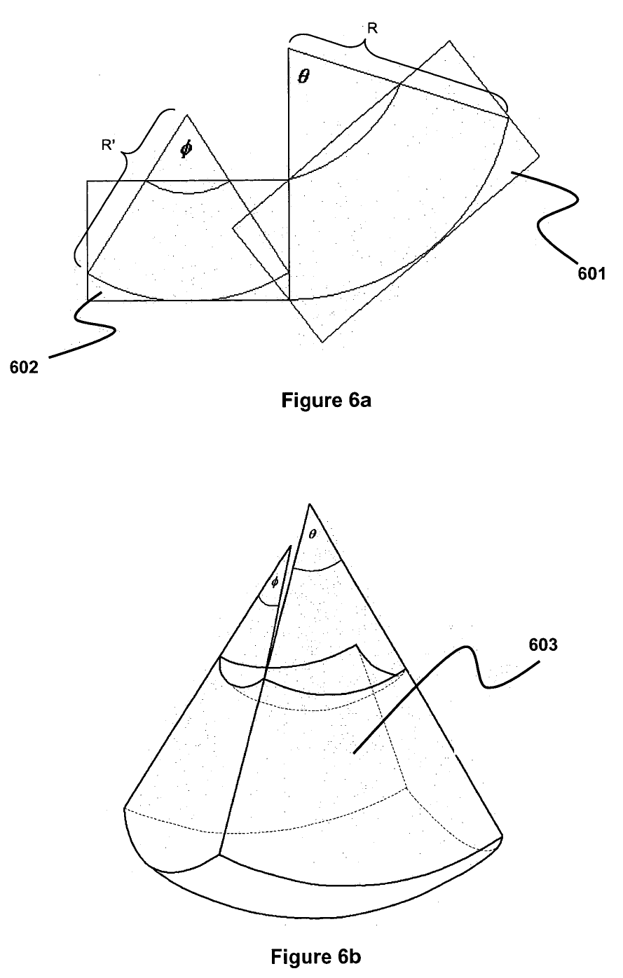

In another embodiment of this invention, using two look-up tables instead of one, ultrasound data in spherical coordinates can be scan-converted to Cartesian coordinates as well. Referring to FIG. 6a, in this case there would be two angles defining the spherical section, i.e., Theta and Phi, and the hardware scan converter of the machine will not be used to generate frames for angle Phi. In this embodiment, a first LUT 601 is used to convert X,Y,X coordinates to X,R,Theta for conversion from the acquisition format for at least one acquisition plane and a second LUT 602 is used to convert X,R,Theta coordinates to R,Theta,Phi for the conversion along the dimension extending out of the acquisition plane. Both acts follow the same approach described in the previous acts, i.e., the spherical coordinate conversion is broken into two perpendicular cylindrical conversion steps. After using the first look-up table, but before using the second one, a wobbling correction step may be used as described above. The resulting spherical section 603 is illustrated in FIG. 6b.

One embodiment of the present invention uses partially scan-converted data, e.g., 2D slices are scan converted by the scan-converter present on the ultrasound apparatus, but a volume acquired by a mechanical or manual wobbling motion of the ultrasound transducer still needs to be converted from Cylindrical to Cartesian space.

Having described embodiments for method for scan conversion of ultrasound data, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

I claim:

1. A system for scan converting ultrasound data from an acquisition format to a display format, the system comprising:
a look-up table having values corresponding to a spatial conversion from the display format to the acquisition format; and
a processor operable to identify acquired ultrasound data as a function of the values and operable to interpolate display values from the identified acquired ultrasound data.

2. The system of claim 1 wherein the values comprise Polar coordinates, the look-up table entries indexed by integer Cartesian coordinates and wherein the processor is operable to bilinearly interpolate from the look-up table values using fractional offsets of Cartesian coordinates.

3. The system of claim 1 wherein the processor is operable to determine display coordinates of interest and identify the acquired ultrasound data by inputting the display coordinates of interest into the look-up table.

4. The system of claim 3 wherein the acquired ultrasound data represents a volume in the acquisition format, wherein the processor is operable to determine display coordinates for a plane through the volume as the display coordinates of interest;
further comprising a display operable to display a two-dimensional image representing the plane in the display format with the display values.

5. The system of claim 3 wherein the acquired ultrasound data represents a volume in the acquisition format, wherein the processor is operable to determine display coordinates for a plurality of rays through the volume as the display coordinates of interest;
further comprising a display operable to display a two-dimensional image of a Volume Rendering of at least a portion of the volume in the display format with the display values.

6. The system of claim 5 wherein each of the display values is a function of an alpha blending of a plurality of acquired ultrasound data values and wherein the processor is operable to limit a number of acquired ultrasound data values blended as a function of a threshold such that scan conversion of other acquired ultrasound data values is avoided.

7. The system of claim 1 further comprising an RGBA look-up table addressed by the display values, the RGBA look-up table operable to output an RGBA value corresponding to the display value.

8. The system of claim 1 wherein the acquired ultrasound data comprises data associated with acquisition by a wobbler transducer array, wherein the values of the look-up table include corrections for shear distortion.

9. The system of claim 1 wherein the look-up table values correspond to the spatial conversion from the display format to the acquisition format for at least one acquisition plane;
further comprising an additional look-up table corresponding to spatial conversion from the display format to the acquisition format across multiple acquisition planes.

10. The system of claim 1 wherein the acquired ultrasound data represents a plurality of scan planes, the acquired ultrasound data of each scan plane in a Cartesian coordinate format, each of the scan planes positioned in the volume in a Polar coordinate format, where the look-up table values correspond to the spatial conversion from the Cartesian coordinate format to the Polar coordinate format relative to the scan plane positions in the volume.

11. The system of claim 1 wherein the processor comprises a graphics processing unit.

12. The system of claim 1 wherein the look-up table values each comprise a set of two fixed-point values, one Boolean Flag, and one Integer Sum, the two fixed-point values being Polar coordinates.

13. The system of claim 12 wherein the Boolean Flag indicates whether the set corresponds to a location outside of scanned region.

14. A method for scan conversion of ultrasound data from an acquisition format to a display format, the method comprising:
(a) identifying acquisition format coordinates with display format coordinates indexed to a look-up table;
(b) interpolating acquisition format coordinates stored in the look-up table; and
(c) interpolating display values from acquired ultrasound data based on the acquisition format coordinates determined in (b).

15. The method of claim 14 wherein (a) comprises:
(a1) inputting Cartesian coordinates into the look-up table; and
(a2) outputting Polar coordinates interpolated from the look-up table in response to (a1).

16. The method of claim 14 further comprising:
(d) determining display coordinates of interest;
wherein (a) comprises inputting the display coordinates of interest into the look-up table.

17. The method of claim 16 wherein the acquired ultrasound data represents a volume in the acquisition format;
wherein (d) comprises determining display coordinates for a plane through the volume as the display coordinates of interest; and
further comprising:
(e) displaying a two-dimensional MPR image representing the plane in the display format as a function of the display values.

18. The method of claim 16 wherein the acquired ultrasound data represents a volume in the acquisition format;
wherein (d) comprises determining display coordinates for a plurality of rays through the volume as the display coordinates of interest; and
further comprising:
(e) displaying a two-dimensional Volume Rendering of at least a portion of the volume in the display format as a function of the display values.

19. The method of claim 18 wherein (e) comprises alpha blending a plurality of acquired ultrasound data values for each of the display values; and
further comprising:
(f) limiting a number of acquired ultrasound data values blended in (e) as a function of a threshold; and
(g) avoiding scan conversion of a plurality of acquired ultrasound data based on (f).

20. The method of claim 14 further comprising:
(d) inputting the display values into an RGBA look-up table; and
(e) outputting RGBA values corresponding to the display values in response to (d).

21. The method of claim 14 further comprising:
(d) acquiring the acquired ultrasound data with a wobbler transducer array;
wherein (a) comprises correcting for shear associated with (d) as a function of the values of the look-up table.

22. The method of claim 14 wherein (a) comprises determining a spatial conversion from the display format to the acquisition format for at least one acquisition plane;
further comprising:
(d) spatially converting from the display format to the acquisition format across multiple acquisition planes with an additional look-up table.

23. The method of claim 14 wherein the acquired ultrasound data represents a plurality of scan planes with the acquired ultrasound data of each scan plane in a Cartesian coordinate format and each of the scan planes positioned in the volume in a Polar coordinate format, wherein (a) comprises spatially converting from the Cartesian coordinate format to the Polar coordinate format relative to the scan plane positions in the volume.

24. The method of claim 14 further comprising:
(d) generating the look-up table as a function of a spatial relationship of a display format with user configured acquisition parameters.

25. The method of claim 14 further comprising:
(d) identifying whether the acquisition format coordinates are outside of scanned region with the look-up table.

26. The method of claim 24 wherein (d) comprises generating a two-dimensional look-up table with acquisition format coordinates for each coordinate of a Cartesian volume.

27. The method of claim 14 further comprising:
(d) Volume Rendering as a function of the display values as a function of time.
